(19) United States
(12) Patent Application Publication
(10) Pub. No.: US 2019/0099137 A1
(43) Pub. Date: Apr. 4, 2019

(54) Intra-Operative Imaging

(71) Applicant: NuVasive, Inc., San Diego, CA (US)

(72) Inventor: Eric Finley, San Diego, CA (US)

(21) Appl. No.: 16/152,361

(22) Filed: Oct. 4, 2018

Related U.S. Application Data
(60) Provisional application No. 62/568,280, filed on Oct. 4, 2017.

(51) Int. Cl. A61B 6/00; A61B 6/12

(52) U.S. Cl. CPC A61B 6/4007; A61B 6/4441; A61B 6/466; A61B 6/12; A61B 2090/3966; A61B 6/542; A61B 6/5235; A61B 6/482; A61B 6/5258

(57) ABSTRACT

A system for medical imaging during a surgical procedure. The system includes an adjustable stand. The system also includes a plurality of emitters coupled to the adjustable stand. The system also includes a receiver configured to capture radiant energy from the plurality of emitters. The system also includes a control unit configured to direct transmission of the radiant energy via the plurality of emitters. The system also includes a display. The system also includes an image processing device in communication with the control unit and the receiver. The image processing device is configured to determine one or more three-dimensional images that correspond to an anatomical structure of a patient based on the data derived from radiant energy captured by the receiver. The image processing device is further configured to provide instructions to display a plurality of images pertaining to the anatomical structure on the display.

### TECHNICAL FIELD

This disclosure relates to medical imaging during surgery.

### BACKGROUND

Intraoperative monitoring is commonly employed during surgeries which involve passing surgical instruments near or through tissues or areas having neural structures which, if contacted, may result in neurological deficit for the patient. For example, spine surgery may be employed to address any number of different spinal disorders. During spine surgery, it is necessary to create an operative corridor extending between an incision site and the spinal column. Depending on the approach or trajectory to the spine (e.g., anterior, posterior, lateral, etc.), different tissues will need to be traversed in order to establish the operative corridor. Further, if a patient's spinal column is manipulated during surgery, soft tissues surrounding the vertebra may be impacted. Regardless of the approach or trajectory, it is helpful to provide image data associated with a surgical site and its surrounding environment to one or more medical professionals.

### SUMMARY

Example systems and methods for medical imaging during a surgical procedure are herein described.

In one embodiment, a system for medical imaging during a surgical procedure comprises an adjustable stand configured to be moved in a plurality of directions. The system includes a plurality of emitters. The plurality of emitters comprises at least a first, second and third emitter coupled to the adjustable stand. The system includes a receiver configured to capture radiant energy from the plurality of emitters, wherein the receiver is coupled to the adjustable stand. The system also includes a control unit configured to direct transmission of the radiant energy via the plurality of emitters. The system includes a display. The system includes an image processing device in communication with the control unit, the receiver and the display

In another embodiment, a method for medical imaging during a surgical procedure is disclosed. The method includes directing transmission of radiant energy via at least a first emitter coupled to an adjustable stand along a first arc of the adjustable stand and at least a second emitter coupled to the adjustable stand along a second arc of the adjustable stand. The method also includes receiving data derived from the radiant energy captured by a receiver coupled to the adjustable stand. The method also includes based on the data derived from the radiant energy, determining one or more three-dimensional images that correspond to an anatomical structure of a patient. The method also includes based on the determined one or more three-dimensional images, providing instructions to display a plurality of images pertaining to the anatomical structure.

In another embodiment, an apparatus for medical imaging during a surgical procedure is disclosed. The apparatus includes an adjustable stand. The apparatus also includes a plurality of emitters configured to transmit radiant energy. The plurality of emitters includes a first emitter coupled to the adjustable stand along a first curvature of the adjustable stand. The plurality of emitters also includes a second emitter coupled to the adjustable stand along a second curvature of the adjustable stand. The plurality of emitters also includes a third emitter coupled to the adjustable stand. The apparatus also includes a receiver configured to capture the radiant energy from the plurality of emitters. The receiver is coupled to the adjustable stand. The apparatus also includes an actuator coupled to the adjustable stand. The apparatus also includes a control unit in communication with the plurality of emitters and the actuator.

### BRIEF DESCRIPTION OF THE DRAWINGS

Many advantages of the present invention will be apparent to those skilled in the art with a reading of this specification in conjunction with the attached drawings, wherein like reference numerals are applied to like elements and wherein:

FIG. 1 is a diagram of an example system for medical imaging during a surgical procedure, according to an embodiment of the present disclosure;

FIG. 2 depicts an example scenario in which the example system of FIG. 1 may be implemented, according to an embodiment of the present disclosure;

FIG. 3 depicts another example scenario in which the example system of FIG. 1 may be implemented, according to an embodiment of the present disclosure;

FIG. 4 depicts another example scenario in which the example system of FIG. 1 may be implemented, according to an embodiment of the present disclosure;

FIG. 5 depicts a portion of an example adjustable stand of FIG. 1, according to an embodiment of the present disclosure;

FIG. 6 depicts a portion of an example adjustable stand of FIG. 1, according to an embodiment of the present disclosure;

FIG. 7 depicts a portion of an example adjustable stand of FIG. 1, according to an embodiment of the present disclosure;

FIG. 8 depicts a portion of an example adjustable stand of FIG. 1, according to an embodiment of the present disclosure;

FIG. 9 depicts an example apparatus for medical imaging during a surgical procedure, according to an embodiment of the present disclosure;

FIG. 10 depicts an example method for medical imaging during a surgical procedure according to an embodiment of the present disclosure.

### DETAILED DESCRIPTION

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

In some embodiments of the present invention, the time and costs associated with surgical imaging may be reduced by using a system for medical imaging during a surgical procedure as described herein. The system may include an adjustable stand that is configured to be moved in a plurality of directions. In one example, the adjustable stand is a C-arm gantry that allows a great degree of flexibility in its positioning.

In one embodiment, the adjustable stand includes a plurality of emitters that act as a distributed source of radiation. The plurality of emitters include various fluoroscopic tubes that are associated with various level of radiation output. In one example, the plurality of emitters are configured to provide a continuous or pulsed output.

In one embodiment, the plurality of emitters include a first set of emitters that are coupled to the adjustable stand along a first axis. The first axis is associated with a first curvature of the adjustable stand.

In one embodiment, the plurality of emitters include a second set of emitters that are coupled to the adjustable stand along a second axis. In this embodiment, the second axis is associated with a second curvature of the adjustable stand.

In one embodiment, the plurality of emitters include a central emitter that is coupled to the adjustable stand at a location associated with the intersection of the first axis and the second axis. In one embodiment, the first axis and the second axis are perpendicular to each other. In one example, each of the plurality of emitters is configured to provide a different dosage of radiant energy. Each of the plurality of emitters can be configured to provide a specific dosage of radiant energy according to the needs of the user. In one example, the central emitter is configured to provide a higher dosage of radiant energy than the other emitters. In another example, all of the emitters are configured to provide the same dosage of radiant energy.

In one embodiment, the plurality of emitters are positioned in different planes relative to each other. In one example, the plurality of emitters are positioned at one or more different angles relative to each other. The use of multiple emitters in different planes and at different angles allows an patient to direct transmission of radiant energy via the plurality of emitters with a single input (e.g., depressing a button associated with operation of the plurality of emitters) and save time associated with determining three-dimensional images corresponding to an anatomical structure of an patient.

In one embodiment, the adjustable stand includes a receiver configured to capture radiant energy from the plurality of emitters. In one example, the receiver is coupled to the adjustable stand at a location a distance from the plurality of emitters. In one example, the receiver is an X-ray image intensifier configured to convert the radiant energy into a visible image. In another example, the receiver is a flat panel detector.

The system for medical imaging also includes a display. In one embodiment, the display is configured to display a plurality of images pertaining to the anatomical structure of the patient receiving treatment. In one example, the plurality of images are determined according to one or more three-dimensional images.

In one embodiment, the system for medical imaging includes a control unit in communication with the plurality of emitters and an image processing device. In one example, the control unit is configured to direct transmission of radiant energy via the plurality of emitters. In one example, the control unit is configured to optimize the selection of which emitters to activate according to a predetermined order. In one example, the control unit is configured to activate a subset of the plurality of emitters. In one scenario, the control unit is configured to modulate the level of radiant energy transmitted by the plurality of emitters. In another scenario, the control unit is configured to activate the plurality of emitters simultaneously.

In one example, based on activation of the plurality of emitters, the image processing device is configured to receive image data derived from the radiant energy captured by the receiver. In this example, upon receipt of the image data, the image processing device is configured to determine one or more three-dimensional images that correspond to the anatomical structure of the patient.

For example, the image processing device compares the image data to a baseline image of the anatomical structure of the patient prior to the surgical procedure. In this example, the baseline image is based on a high dosage of radiant energy and thus provides a clear view of the anatomical structure. Further, the baseline image may contain data that corresponds to one or more markers (e.g., tracking pins) coupled to the anatomical structure.

In this example, the image processing device is configured to determine the corresponding sagittal and axial views of the baseline image based on the data derived from the radiant energy. In one scenario, the image processing device is configured to compare the data derived (i.e., image data) from the radiant energy to the baseline image until a threshold is satisfied. In this scenario, upon satisfaction of the threshold, the image processing device is configured to determine one or more three-dimensional images of an anatomical structure of a patient. Further, based on the determined one or more three-dimensional images, the image processing device is configured to provide instructions to display a plurality of images pertaining to the anatomical structure on the display. In one example, the plurality of images include an axial view and a sagittal view of the anatomical structure.

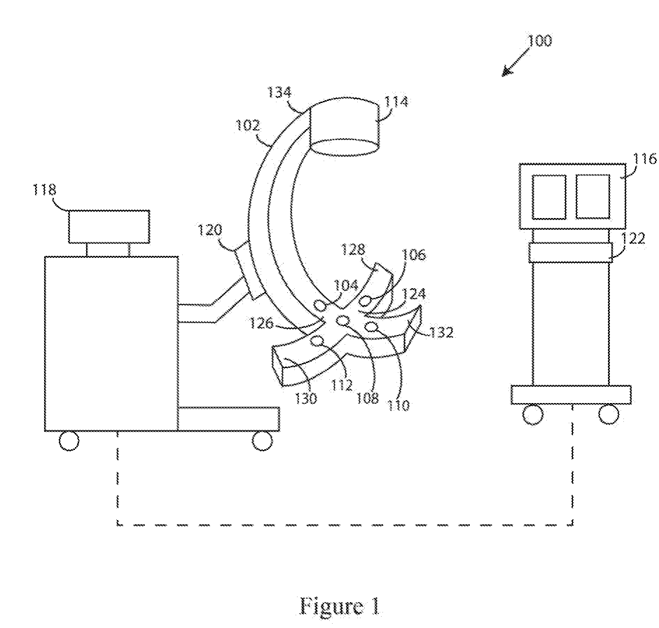

Referring now to the figures, FIG. 1 is a diagram of an example system 100 for medical imaging during a surgical procedure. The example system 100 includes an adjustable stand 102, a control unit 118, a display 116, and an image processing device 122. In one embodiment, the control unit 118 and the image processing device 122 communicate via one or more communication channels.

In one example, the adjustable stand 102 is a C-arm gantry. The adjustable stand 102 includes a plurality of emitters 104, 106, 108, 110, and 112 and a receiver 114. In one embodiment, the plurality of emitters 104, 106, 108, 110, and 112 are selected from one or more various flat panel detectors and X-ray image intensifiers. While the exemplary embodiment shown in FIG. 1 includes emitters 104, 106, 108, 110, and 112, in a generally cruciform configuration, it is also contemplated that the adjustable stand may include any combination of three or more emitters in varying spatial relationships.

According to the exemplary embodiment illustrated in FIG. 1, a first set of emitters 106 and 112 are coupled along a first arc 124 of the adjustable stand 102. As shown in FIG. 1, emitter 106 is coupled to the adjustable stand 102 on a first arm 128 and emitter 112 is coupled to the adjustable stand 102 on a second arm 130 extending opposite of the first arm 128. In one embodiment, the first arm 128 and the second arm 130 are arcuate. A second set of emitters 104 and 110 are coupled along a second arc 126 of the adjustable stand 102. Emitter 110 is coupled to the adjustable stand 102 at a first end 132 of the adjustable stand 102. As shown in FIG. 1, emitter 108 is located along the intersection of the first arc 124 and the second arc 126. The plurality of emitters 104, 106, 108, 110, and 112 are configured to transmit radiant energy towards the receiver 114. It is known that the radiant energy emanated from a radiation source is conical so that the field of exposure may be varied by moving the adjustable stand 102 closer to or away from a patient.

In one embodiment, the control unit 118 includes a control panel (not shown) through which a radiology technician can control the operation of the adjustable stand 102. The control panel permits the technician to “shoot a picture” of the surgical site at a surgeon's direction, control the radiation dose, and initiate transmission of radiant energy from the plurality of emitters 104, 106, 108, 110, and 112.

According to one aspect, the system 100 includes an actuator 120 coupled to the adjustable stand 102. In one embodiment, the control unit 118 communicates with the actuator 120 through a wired or wireless interface. In one example, the actuator 120 is configured to rotate the adjustable stand 102 in one or more directions based on instructions received from the control unit 118 for different viewing angles of the surgical site. Thus, the position of the receiver 114 and the plurality of emitters 104, 106, 108, 110, and 112 relative to a patient, and more particularly relative to the surgical site of interest, may change during a procedure as needed by the surgeon or radiologist.

The receiver 114 is configured to capture radiant energy from the plurality of emitters 104, 106, 108, 110, and 112 and convert the radiant energy into image data. The receiver is coupled to the adjustable stand 102 at a second end 134 of the adjustable stand 102. Further, in one example, the receiver 114 is configured to provide the image data to the image processing device 122.

According to an exemplary embodiment, the image processing device 122 is configured to analyze the image data from the receiver 114 in order to determine one or more three-dimensional images that correspond to the anatomical structure of a patient. In one example, the image processing device 122 includes a digital memory associated therewith and a processor for executing digital and software instructions. In one example, the image processing device 122 incorporate a frame grabber that uses frame grabber technology to create a digital image for projection on display 116.

In one embodiment, the display 116 is configured to receive and display image data corresponding to the anatomical structure of the patient. In one scenario, the display 116 is positioned for interactive viewing by the surgeon during the procedure. In one scenario, the display 116 is used to show images from two views, such as a lateral and AP. In one example, an input device (not shown), such as a keyboard or a touch screen, is coupled to the display 116 and allows a surgeon or other operating room staff to select and manipulate the on-screen images.

In one scenario, the image processing device 122 receives information indicative of the anatomical structure prior to the surgical procedure. For example, the information indicative may be one or more “full dose” X-ray images of the anatomical structure or a three-dimensional model of the anatomical structure. In this scenario, the image processing device 122 is configured to compare the data derived (based on a low dose X-ray) from the radiant energy during the surgical procedure to the information indicative of the anatomical structure prior to the surgical procedure to determine a match between both sets of image data.

To do so, the image processing device 122 utilizes various algorithms to find a statistically meaningful match between the low dose image and the full dose image, for example. In at least one approach, comparisons can be made at predetermined locations associated with the low dose images and the full dose images. For example, pixel comparisons can be concentrated in regions of the images believed to provide a greater likelihood of a relevant match. By way of example, a region of the image that may provide a greater likelihood of a relevant match may be a region that includes image data representative of a marker such as metallic tracking pin.

In one scenario, based on not finding a meaningful match, the image processing device 122 is configured to determine that the data derived from the radiant energy does not satisfy a threshold. In this scenario, the image processing device 122 is configured to provide an instruction to the control unit 118 to cause the actuator 120 to rotate the adjustable stand 102 to a first position.

In one scenario, after the adjustable stand 102 has been rotated to the first position, the control unit 118 is configured to activate the plurality of emitters 104, 106, 108, 110, and 112 in order to transmit radiant energy. In this scenario, the image processing device 122 is configured to receive the data derived from the radiant energy captured by the receiver while the adjustable stand 102 is in the first position. Based on a new comparison of the data derived from the radiant energy captured at the first position and the information indicative of the anatomical structure prior to the surgical procedure, the image processing device 122 is configured to determine one or more three-dimensional images that correspond to the anatomical structure of the patient. Further, the image processing device 122 is configured to provide instructions to display a plurality of images pertaining to the anatomical structure on the display 116.

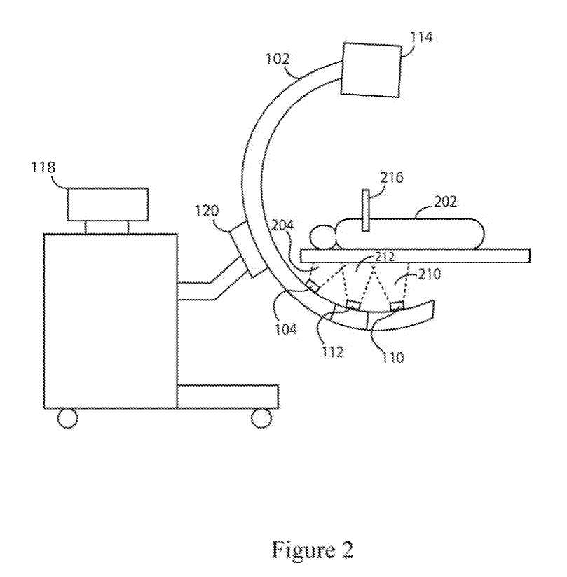

FIG. 2 depicts an example depiction of a patient 202 undergoing a surgical procedure that includes surgical tool 216. The adjustable stand 102, the control unit 118, and the actuator 120 from FIG. 1 are shown in FIG. 2.

Referring to FIG. 2, the transmission of radiant energy 204, 210, and 212 via emitters 104, 110, and 112 is targeted at an anatomical structure of the patient 202. In one example, the anatomical structure consists of one or more vertebrae of the spine.

In one scenario, the control unit 118 is configured to receive a command to activate emitters 104, 110, and 112 (including emitters 106 and 108 from FIG. 1 which are not shown) based on an input from a technician. The receiver 114 is configured to convert the radiant energy 204, 210, and 212 captured into image data. Depending on the surgical procedure, the image data may contain one or more artifacts corresponding to surgical tools such as surgical tool 216. In one example, the receiver 114 is configured to provide the image data to the image processing device 122 for further processing.

In this scenario, the image processing device 122 is configured to detect one or more artifacts from the data derived from the radiant energy 204, 210, and 212. The image processing device 122 is configured to compare the detected artifact to image data associated with one or more surgical tools stored in a database. Based on a match of the detected artifact to the image data, the image processing device 122 is configured to determine a virtual representation of the surgical tool 216. In one scenario, the image processing device 122 is configured to provide instructions to overlay the virtual representation of the surgical tool 216 on a plurality of images pertaining to the anatomical structure of patient 202.

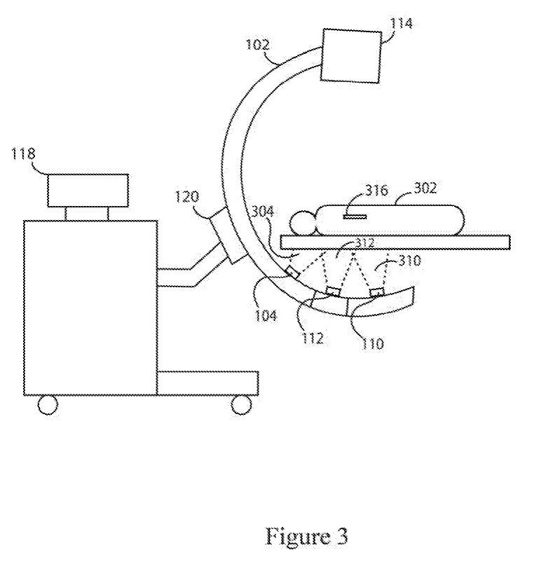

FIG. 3 depicts another example depiction of a patient 302 undergoing a surgical procedure that includes a marker 316 coupled to the anatomical structure of the patient 302. The adjustable stand 102, the control unit 118, and the actuator 120 from FIG. 1 are shown in FIG. 3.

In one scenario, the control unit 118 may be configured to activate emitters 104, 110, and 112 (including emitters 106 and 108 from FIG. 1 that are not shown) based on an input from a technician. Referring to FIG. 3, the transmission of radiant energy 304, 310, and 312 via emitters 104, 110, and 112 is targeted at an anatomical structure of the patient 302. In one embodiment, the receiver 114 is configured to capture the radiant energy 204, 210, and 212 and convert the radiant energy 204, 210, and 212 into image data. Depending on the surgical procedure, the image data may contain one or more artifacts corresponding to markers, such as marker 316, used to identify a location and orientation of the spine within an image. In one example the marker 316 may be a metallic tracking pin. In one example, the receiver 114 is configured to provide the image data to the image processing device 122 for further processing.

In this scenario, the image processing device 122 is configured to detect one or more artifacts from the data derived from the radiant energy 304, 310, and 312. The image processing device 122 may compare the detected artifact to image data associated with one or more surgical markers stored in a database. The database may be local or remote to the image processing device 122. Based on the comparison satisfying a threshold, the image processing device 122 is configured to determine one or more three-dimensional images that correspond to the anatomical structure of the patient 302, for example.

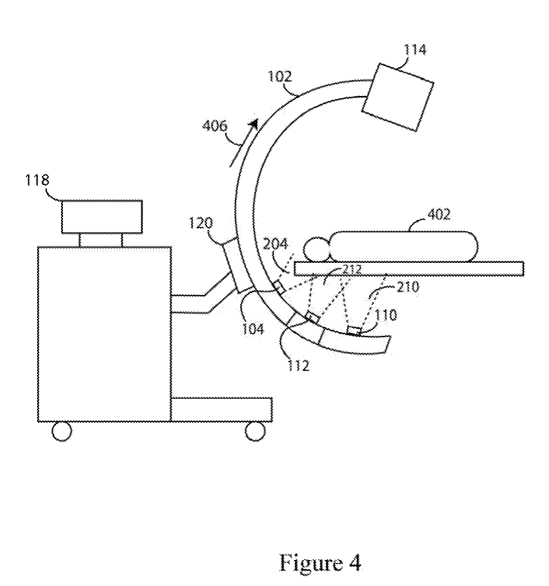

FIG. 4 is a side view of adjustable stand 102 from FIG. 1 and another example scenario with a patient 402 undergoing a surgical procedure.

Referring to FIG. 4, the adjustable stand 102 from FIG. 1 is rotated by a predetermined amount along a direction 406. In one scenario, during the rotation of the adjustable stand 102, emitters 104, 112, 110 (in addition to emitters 108 and 106 that are not shown) are activated to provide radiant energy 204, 210, and 212 towards receiver 114. In this scenario, the control unit 118 from FIG. 1 is configured to provide instructions to the actuator 120 to cause the adjustable stand 102 to rotate and activate the plurality of emitters 104, 106, 108, 110, and 112 during the rotation of the adjustable stand 102.

In one scenario, the control unit 118 is further configured to modulate a level of radiant energy associated with the plurality of emitters 104, 106, 108, 110, and 112 during the rotation of the adjustable stand 102. For example, as the rotation is occurring, the image processing device 122 of FIG. 1 receives image data captured by receiver 114. In one scenario, once the image processing device 122 determines sufficient information pertaining to an area of interest from the image data captured by the receiver 114, the image processing device 122 is configured to provide a signal to the control unit 118 that causes an increase or decrease in the level of radiant energy transmitted by the plurality of emitters 104, 106, 108, 110, and 112 during the rotation of the adjustable stand 102.

Further, the image processing device 122 is configured to compare the data derived from the radiant energy during the rotation of the adjustable stand 102 to the information indicative of the anatomical structure prior to the surgical procedure and determine that a threshold is satisfied according to one or more algorithms described above, for example. In this scenario, the image processing device 122 is configured to provide a stop signal to the control unit 118. Based on receipt of the stop signal, the control unit 118 is configured to cause the actuator 120 to stop rotation along direction 406 and maintain a position of the adjustable stand 102. Furthermore, the control unit 118 may also be configured to end the transmission of radiant energy via the plurality of emitters 104, 106, 108, 110, and 112 according to the received stop signal.

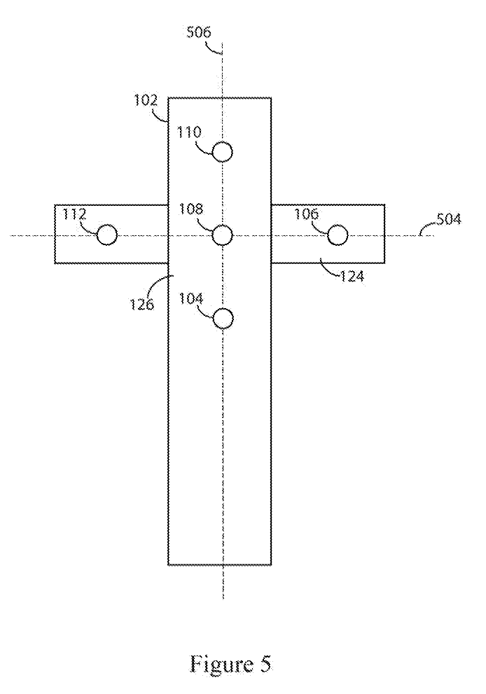

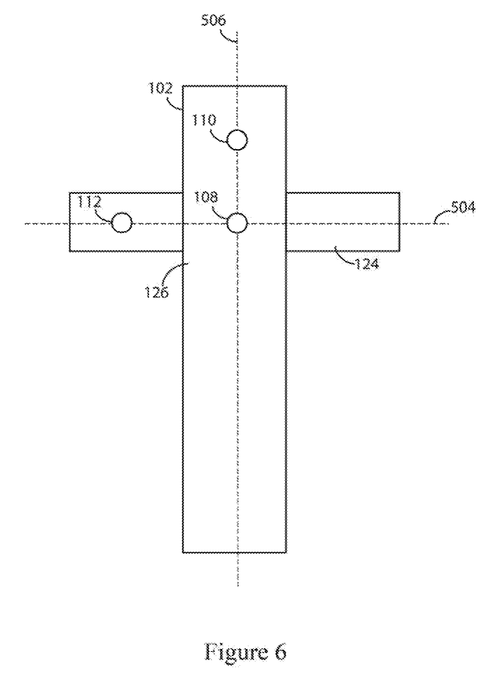

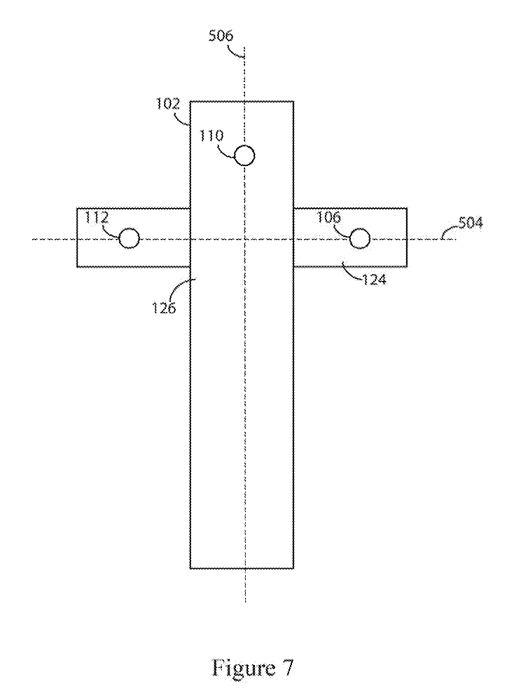

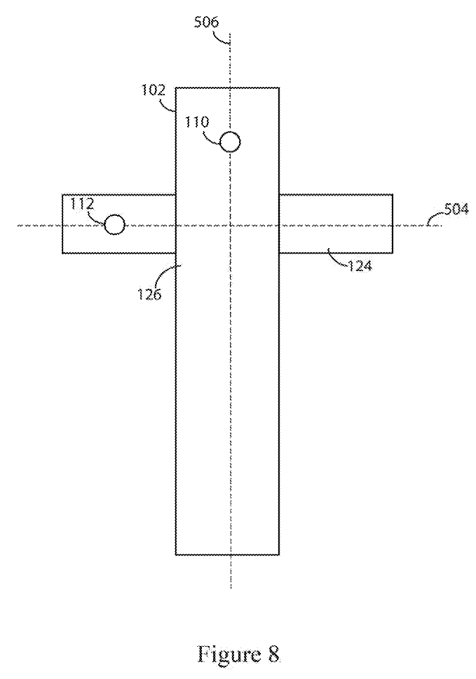

FIG. 5 depicts a partial view of the first end 132 of the adjustable stand 102 of FIG. 1. As shown in FIG. 5, a first set of emitters 106 and 112 are coupled to the adjustable stand 102 along a first axis 504. A second set of emitters 104 and 110 are coupled to the adjustable stand 102 along a second axis 506. A central emitter 108 is coupled to the adjustable stand 102 at a location associated with the intersection of the first axis 504 and the second axis 506. Although the first axis 504 and the second axis 506 are perpendicular to one another, other angles of intersection associated with the first axis 504 and the second axis 506 are envisioned. Alternative spatial arrangements and numbers of emitters are also contemplated and may be used to achieve the functionality described herein. Exemplary alternative embodiments may include, but are not limited to, those shown in FIGS. 6, 7, and 8, depicting partial views of the first end 132 of the adjustable stand 102 of FIG. 1.

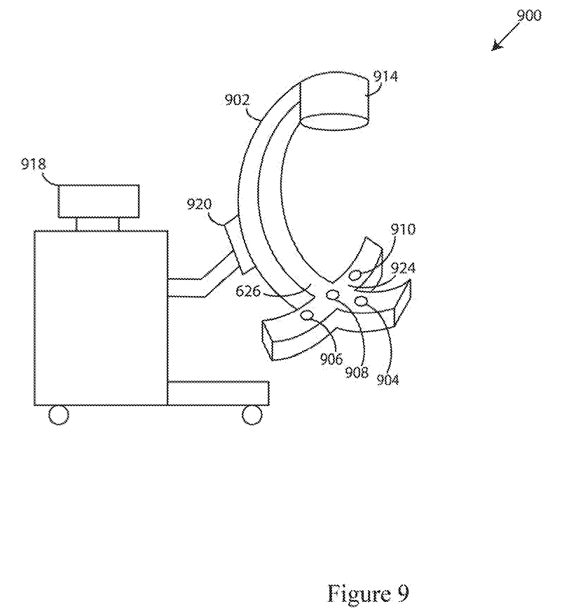

FIG. 9 depicts an apparatus 900 for medical imaging during a surgical procedure that may be used to achieve the functionality associated with corresponding components of system 100 of FIG. 1. The apparatus 900 includes an adjustable stand 902, an actuator 920 coupled to the adjustable stand 902, and a control unit 918. The apparatus 900 includes a plurality of emitters 904, 906, 908, and 910 configured to transmit radiant energy. The plurality of emitters 904, 906, 908, and 910 are configured to operate at a plurality of levels of radiant energy based on one or more instructions from the control unit 918.

As shown in FIG. 9, emitter 904 is coupled to the adjustable stand 902 along a first curvature 924 of the adjustable stand 902. Emitter 904 is coupled to the adjustable stand 902 along a second curvature 926 of the adjustable stand 902. Emitter 908 is coupled to the adjustable stand 902 along the intersection of the first curvature 924 and the second curvature 926 of the adjustable stand 902. Emitter 910 is coupled to the adjustable stand 902 along the first curvature 924 of the adjustable stand 902.

In one or more embodiments, the apparatus 900 contains more or less emitters than those shown in FIG. 9. In one embodiment, the apparatus 900 only includes emitters 904, 906, and 908. In another embodiment, the apparatus 900 only includes emitters 904, 908, and 910. Based on the use of less emitters, the apparatus 900 is configured to produce less scatter of radiant energy.

The receiver 914 is coupled to the adjustable stand 902 and configured to capture the radiant energy from the plurality of emitters 904, 906, 908, and 910. Further, the receiver 914 is configured to transmit image data based on the radiant energy to an imaging unit (e.g., image processing device 122 of FIG. 1) for further processing of the image data.

The apparatus 900 also includes an actuator 920 coupled to the adjustable stand 902. The actuator 920 is configured to rotate the adjustable stand 920 based on one or more instructions received from the control unit 918. In one or more embodiments, the actuator 920 comprises an energy source that is based on electric current, hydraulic fluid, or a pneumatic pressure. In one embodiment, actuator 920 is a mechanical actuator that is dependent on a user to exert a force to provide movement of the adjustable stand 902.

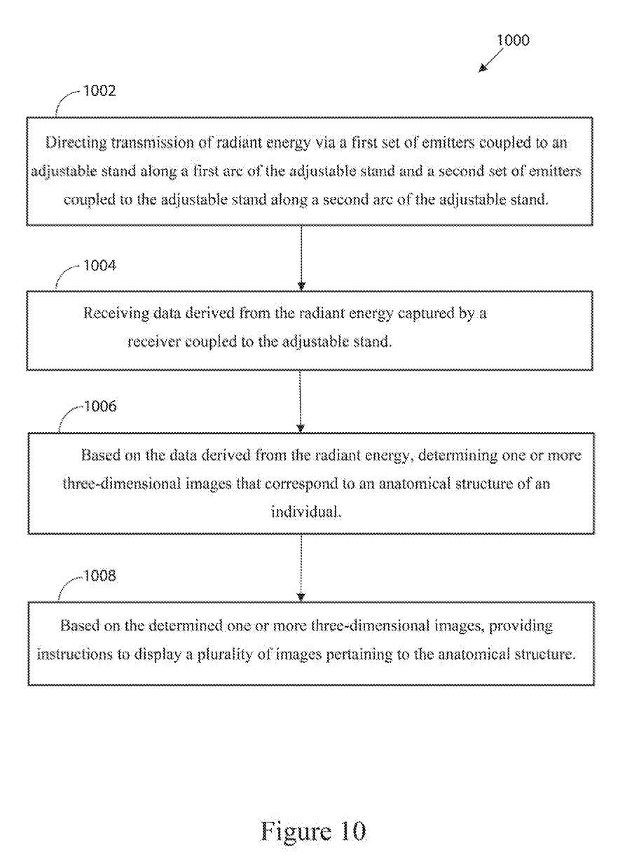

FIG. 10 is a flow diagram of an example method for medical imaging during a surgical procedure, in accordance with at least one embodiment described herein. Although the blocks in each figure are illustrated in a sequential order, the blocks may in some instances be performed in parallel, and/or in a different order than those described therein. Also, the various blocks may be combined into fewer blocks, divided into additional blocks, and/or removed based upon the desired implementation.

In addition, the flow diagram of FIG. 10 shows the functionality and operation of one possible implementation of the present embodiment. In this regard, each block may represent a module, a segment, or a portion of program code, which includes one or more instructions executable by a processor for implementing specific logical functions or steps in the process. The program code may be stored on any type of computer readable medium, for example, such as a storage device including a disk or hard drive. The computer readable medium may include non-transitory computer-readable media that stores data for short periods of time, such as register memory, processor cache, or Random Access Memory (RAM), and/or persistent long term storage, such as read only memory (ROM), optical or magnetic disks, or compact-disc read only memory (CD-ROM), for example. The computer readable media may be able, or include, any other volatile or non-volatile storage systems. The computer readable medium may be considered a computer readable storage medium, a tangible storage device, or other article of manufacture, for example.

Alternatively, each block in FIG. 10 may represent circuitry that is wired to perform the specific logical functions in the process. Illustrative methods, such as those shown in FIG. 10, may be carried out in whole in or in part by a component or components in the cloud and/or system 100 of FIG. 1. However, it should be understood that the example methods may instead be carried out by other entities or combinations of entities (i.e., by other computing devices and/or combination of computer devices), without departing from the scope of the invention. For example, functions of the method of FIG. 10 may be fully performed by a computing device (or components of a computing device such as one or more processors), or may be distributed across multiple components of the computing device, across multiple computing devices (e.g., control unit 118 and image processing device 122 of FIG. 1), and/or across a server.

Referring to FIG. 10, an example method 1000 for medical imaging during a surgical procedure may include one or more operations, functions, or actions as illustrated by blocks 1002-1008. In one embodiment, the method 1000 is implemented in whole or in part by the system 100 of FIG. 1.

As shown by block 1002, the method 1000 includes directing transmission of radiant energy via a first set of emitters coupled to an adjustable stand along a first arc of the adjustable stand and a second set of emitters coupled to the adjustable stand along a second arc of the adjustable stand.

As shown by block 1004, the method 1000 also includes receiving data derived from the radiant energy captured by a receiver coupled to the adjustable stand.

Block 1004 may also, or instead, include receiving information indicative of the anatomical structure prior to the surgical procedure. Block 1004 may further include comparing the data derived from the radiant energy during the surgical procedure to the information indicative of the anatomical structure prior to the surgical procedure. Based on the comparison, block 1004 may include determining the data derived from the radiant energy does not satisfy a threshold. Based on not satisfying the threshold, block 1004 may include rotating the adjustable stand, via an actuator, and activating the first set of emitters and the second set of emitters during the rotation of the adjustable stand. In one scenario, the level of energy associated with the first set of emitters and the second set of emitters during the rotation of the adjustable stand may be modulated.

In another example, block 1004 may also, or instead, include comparing the data derived from the radiant energy during the rotation of the adjustable stand to the information indicative of the anatomical structure prior to the surgical procedure. Based on the comparison, block 1004 may include determining that the data derived from the radiant energy satisfies a threshold. A stop signal may be provided to the actuator and the plurality of emitters, wherein the stop signal is configured to (i) cause the actuator to maintain a current position of the adjustable stand and (ii) end the transmission of radiant energy via the plurality of emitters.

In another example, block 1004 may also, or instead, include detecting one or more artifacts from the data derived from the radiant energy. The one or more artifacts may be compared to image data associated with one or more surgical tools. Based on a match of the one or more artifacts to the image data, a virtual representation of a given tool of the one or more surgical tools may be overlaid on the plurality of images pertaining to the anatomical structure.

In another example, block 1004 may also, or instead, include receiving information indicative of the anatomical structure prior to the surgical procedure. Block 1004 may further include comparing the data derived from the radiant energy during the surgical procedure to the information indicative of the anatomical structure prior to the surgical procedure. Based on the comparison, block 1004 may include determining that the data derived from the radiant energy does not satisfy a threshold. Based on not satisfying the threshold, block 1004 may include rotating the adjustable stand, via the actuator, to a first position and activating the plurality of emitters at the first position.

As shown by block 1006, the method 1000 also includes based on the data derived from the radiant energy, determining one or more three-dimensional images that correspond to an anatomical structure of a patient.

In one example, block 1006 may also, or instead, include receiving information indicative of a marker coupled to the anatomical structure of the patient, wherein the marker is a metallic tracking pin. Block 1006 may further include comparing the data derived from the radiant energy to the information indicative of the marker coupled to the anatomical structure. Based on the comparison satisfying a threshold, block 1006 may include determining one or more three-dimensional images that correspond to the anatomical structure of a patient.

As shown by block 1008, the method 1000 also includes based on the determined one or more three-dimensional images, providing instructions to display a plurality of images pertaining to the anatomical structure.

In one example, block 1008 may also, or instead, include detecting one or more artifacts from the data derived from the radiant energy. Block 1008 may further include comparing the one or more artifacts to image data associated with one or more surgical tools. Based on a match of the one or more artifacts to the image data, block 1008 may include overlaying a virtual representation of a given tool of the one or more surgical tools on the plurality of images pertaining to the anatomical structure.

Any of the features or attributes of the above described embodiments and variations can be used in combination with any of the other features and attributes of the above described embodiments and variations as desired. Various modifications, additions and other alternative embodiments are possible without departing from the true scope and spirit. The embodiments presented herein were chosen and described to provide an illustration of various principles of the present invention and its practical application to thereby enable one of ordinary skill in the art to utilize the invention in various embodiments and with various modifications as are suited to the particular use contemplated. All such modifications and variations are within the scope of the present invention as determined by the appended claims when interpreted in accordance with the benefit to which they are fairly, legally, and equitably entitled.

What is claimed is:

1. A system for medical imaging during a surgical procedure, comprising:
an adjustable stand having a first end and a second end, the first end extending along a first axis, wherein the adjustable stand includes a first arm extending along a second axis and proximate the first end;
a plurality of emitters comprising:
a first emitter coupled to the adjustable stand at the first end;
a second emitter coupled to the adjustable stand on the first arm; and a
a third emitter coupled to the adjustable stand;
a receiver configured to capture radiant energy transmitted from the plurality of emitters, wherein the receiver is coupled to the adjustable stand;
a control unit configured to direct transmission of the radiant energy via the plurality of emitters;
a display; and
an image processing device in communication with the control unit, the receiver, and the display.

2. The system of claim 1, wherein the system further includes an actuator coupled to the adjustable stand and configured to change a position of the adjustable stand.

3. The system of claim 2, wherein the actuator is configured to cause rotation of the adjustable stand.

4. The system of claim 2, wherein the actuator is in communication with the control unit.

5. The system of claim 1, further including a second arm extending opposite the first arm.

6. The system of claim 5, wherein the first and second arms are arcuate.

7. The system of claim 1, wherein the receiver is coupled to the adjustable stand at the second end.

8. The system of claim 5, wherein the plurality of emitters includes:
a first set of emitters coupled to the adjustable stand on at least one of the first or second arms;
a second set of emitters coupled to the adjustable stand proximate the first end of the adjustable stand.

9. The system of claim 8, wherein the first set of emitters includes two emitters.

10. The system of claim 9, wherein the second set of emitters includes two emitters.

11. The system of claim 1, wherein the third emitter is coupled to the adjustable stand at a location associated with an intersection of the first axis and the second axis;

12. A method for medical imaging during a surgical procedure, the method comprising:
directing transmission of radiant energy via a first emitter coupled to an adjustable stand along a first arc of the adjustable stand and a second emitter coupled to the adjustable stand along a second arc of the adjustable stand;
receiving data derived from the radiant energy captured by a receiver coupled to the adjustable stand;
based on the data derived from the radiant energy, determining one or more three-dimensional images that correspond to an anatomical structure of an patient; and
based on the determined one or more three-dimensional images, providing instructions to display a plurality of images pertaining to the anatomical structure.

13. The method of claim 12, further including directing transmission of radiant energy via a third emitter coupled to the adjustable stand and located at an intersection of the first arc of the adjustable stand and the second arc of the adjustable stand.

14. The method of claim 12, further including directing transmission of a radiant energy via a third emitter coupled to the adjustable stand along the first arc of the adjustable stand.

15. The method of claim 12, further comprising:
receiving information indicative of the anatomical structure prior to the surgical procedure;
comparing the data derived from the radiant energy during the surgical procedure to the information indicative of the anatomical structure prior to the surgical procedure;
based on the comparison, determining the data derived from the radiant energy does not satisfy a threshold; and
rotating the adjustable stand and activating at least one of the first emitter and the second emitter during the rotation of the adjustable stand.

16. The method of claim 15, further comprising:
modulating a level of energy associated with the at least one of the first emitter or the second emitter during rotation of the adjustable stand.

17. The method of claim 12, further comprising:
comparing the data derived from the radiant energy during rotation of the adjustable stand to the information indicative of the anatomical structure prior to the surgical procedure;
based on the comparison, determining that the data derived from the radiant energy satisfies a threshold; and
providing a stop signal to an actuator coupled to the adjustable stand, to the first emitter, and to the second emitter, wherein the stop signal is configured to (i) cause the actuator to maintain a current position of the adjustable stand and (ii) end the transmission of radiant energy via the first and second emitters.

18. The method of claim 12, further comprising:
detecting one or more artifacts from the data derived from the radiant energy;
comparing the one or more artifacts to image data associated with one or more surgical tools; and
based on a match of the one or more artifacts to the image data, overlaying a virtual representation of a given tool of the one or more surgical tools on the plurality of images pertaining to the anatomical structure.

19. The method of claim 12, wherein determining the one or more three-dimensional images that correspond to the anatomical structure of the patient further comprises:
receiving information indicative of a marker coupled to the anatomical structure of the patient, wherein the marker is a metallic tracking pin;
comparing the data derived from the radiant energy to the information indicative of the marker coupled to the anatomical structure; and
based on the comparison satisfying a threshold, determining the one or more three-dimensional images that correspond to the anatomical structure of a patient.

20. The method of claim 12, further comprising:
receiving information indicative of the anatomical structure prior to the surgical procedure;
comparing the data derived from the radiant energy during the surgical procedure to the information indicative of the anatomical structure prior to the surgical procedure;
based on the comparison, determining the data derived from the radiant energy does not satisfy a threshold; and
rotating the adjustable stand to a first position and activating the first and second emitters at the first position.

21. The method of claim 12, further comprising:
detecting one or more artifacts from the data derived from the radiant energy,
comparing the one or more artifacts to image data associated with one or more surgical tools, and
based on a match of the one or more artifacts to the image data, overlaying a virtual representation of a given tool of the one or more surgical tools on the plurality of images pertaining to the anatomical structure.

22. An apparatus for medical imaging during a surgical procedure, comprising:
an adjustable stand;
a plurality of emitters configured to transmit radiant energy, the plurality of emitters comprising:
a first emitter coupled to the adjustable stand along a first curvature of the adjustable stand;
a second emitter coupled to the adjustable stand along a second curvature of the adjustable stand; and
a third emitter coupled to the adjustable stand along the first curvature;
a receiver configured to capture the radiant energy from the plurality of emitters, wherein the receiver is coupled to the adjustable stand;
an actuator coupled to the adjustable stand; and
a control unit in communication with the plurality of emitters and the actuator.

23. The apparatus of claim 22, wherein the plurality of emitters are configured to operate at a plurality of levels of radiant energy.

24. The apparatus of claim 23, the plurality of emitters further comprises a fourth emitter coupled to the adjustable stand along the first curvature of the adjustable stand.

25. The apparatus of claim 24, wherein the plurality of emitters further comprises a fifth emitter coupled to the adjustable stand along the second curvature of the adjustable stand.

26. The apparatus of claim 22, wherein the third emitter is coupled to the adjustable stand at an intersection of the first curvature and the second curvature.
